(19) United States
(12) Patent Application Publication
(10) Pub. No.: US 2004/0059376 A1
(43) Pub. Date: Mar. 25, 2004

(54) Device for the introduction and fashioning of skin clips

(76) Inventor: Helmut Breuniger, Toebingen (DE)

Correspondence Address: Breneman & Georges, 3150 Commonwealth Avenue, Alexandria, VA, 22305, US

(21) Appl. No.: 10/470,710

(22) PCT Filed: Dec. 22, 2001
(86) PCT No.: PCT/DE01/04876

(30) Foreign Application Priority Data
Jan. 31, 2001 (DE) 101 04 532.8

(51) Int. Cl. A61B 17/08

(52) U.S. Cl. 606/216

(57) ABSTRACT

A device is proposed for the introduction and shaping of skin clips which, in the initial state, have two side arms which are parallel to each other and to the direction of introduction, and an intermediate piece connecting the side arms, the aim being to reduce the extent of crushing of tissue and the negative consequences thereof upon clipping, and to make it easier to remove the skin clips after healing of the wound. According to the invention, this is achieved by the fact that, in the shaped final state of the skin clips, the deviation of the side arms from the direction of introduction is less than 90°.

The invention relates to a device for the introduction and shaping of skin clips according to the preamble of claim 1.

To connect wound margins, skin clips have hitherto been used which have two side arms and an intermediate piece connecting the side arms. The side arms are at right angles to the intermediate piece and are driven, using a known clip apparatus, into the skin on both sides of the wound, while the wound margins are fixed by suitable measures. The two end areas of the intermediate piece are then bent at right angles by pressing a U-shaped shaping stamp over a counterstay lying on the wound. The two previous side arms of the skin clip are likewise bent in the process and come to lie substantially parallel to the middle, unbent area of the intermediate piece within the skin.

As a result of the extreme deformation of the clip in the skin and the associated pressing-in of the shaping stamp with its two lateral areas into the surface of the skin, this shaping procedure causes enormous crushing of the tissue lying in this area. This crushing of tissue leads to pronounced scar formation during the subsequent healing process. Moreover, the skin clips which have been bent in this way can be removed only with a special instrument, because the considerably deformed skin clip has to be deformed once again in order to remove it.

The object of the invention, by contrast, is to make available a device and a method for introduction and shaping of skin clips, the aim being to reduce the extent of tissue crushing and the negative consequences thereof and to make it easier to remove the skin clips after healing of the wound.

Starting out from a device of the type mentioned by way of introduction, this object is achieved by the characterizing features of claim 1.

Advantageous embodiments and developments of the invention are made possible by the measures specified in the dependent claims.

Accordingly, a device according to the invention is distinguished by the fact that, in the shaped final state of the skin clip, the deviation of the side arms from the direction of introduction is less than 90°. Such a configuration can be achieved with much reduced crushing of tissue and with a reduction in the disadvantageous consequences cited above. Moreover, a skin clip secured in this way is much easier to remove.

In an advantageous development of the invention, a deformation of the intermediate piece is carried out in order to produce this orientation of the side arms. The deformation of the intermediate piece permits shaping of the skin clip essentially above the skin, which in turn serves to reduce crushing of tissue.

In a further particular embodiment of the invention, at least one counterstay is provided. Such a counterstay, on which the skin clip abuts while being introduced into the skin, is able to take up the pressure needed for deformation of the clip, with the result that the skin lying underneath it is largely spared from this pressure.

At least two shaping zones are preferably provided on the shaping stamp of the device on both sides of the counterstay. In this way, it is possible to deform the intermediate piece, after it has struck against the counterstay, by pressure exerted in the direction of introduction of the skin clip and without exerting pressure on the skin lying underneath it. Upon this deformation of the intermediate piece, the side arms of the skin clip which were previously parallel to the direction of introduction are brought into the desired position.

The counterstay which takes up the pressure of the shaping stamp upon deformation of the skin clip is preferably designed as a mandrel. Such a mandrel makes it possible to kink the intermediate piece of the skin clip upon deformation. The kinking of the elongate intermediate piece of the skin clip outside the skin can be done with much less deformation pressure than can a deformation which takes place partially within the skin. This measure accordingly serves in turn to reduce crushing of the skin.

The shaping stamp is preferably V-shaped. This V-shape provides for a central kink in the final shape of the skin clip, the travel of the shaping stamp being limited by the fact that the apex of the V profile strikes against the counterstay, which is preferably designed as a mandrel, as indicated above. In principle, however, such a counterstay can also be of another design, for example adapted to the profile of the shaping stamp. In the case of a V-shaped stamp, the counterstay could accordingly also have a complementary V profile.

The cited illustrative embodiments which prefer a central kink in the intermediate piece of the skin clip are not to be understood as being restrictive. In principle, it would also be possible to deform the intermediate piece with a continuous profile in such a way that the side arms of the skin clip adopt the desired position. It would likewise be possible for the intermediate piece to be kinked several times so as to obtain a polygon progression.

However, in the preferred embodiment, which can be achieved with comparatively little outlay, the skin clip is provided with an intermediate piece which is straight in the initial state and kinked in the shaped final state. The central kink is to be obtained with a minimum of deformation pressure by means of the pressurizing zones of the shaping stamp extending into the edge areas of the intermediate piece of the skin clip so as to obtain a lever action on the intermediate piece of the skin clip.

Advantageously, a magazine is additionally provided for a plurality of skin clips in the initial state, by which means a multiplicity of skin clips can be applied successively in one operation without “reloading” the clip apparatus, as is necessary when clipping wounds in the usual way.

The shaping of the skin clip is in this case advantageously chosen in such a way that the deviation of the side arms, obtained by deformation above the skin surface, from the direction of introduction into the skin is less than 45°. In principle, in order to achieve the advantages according to the invention, a position of the side arms is sought which comes as close as possible to the original state with the side arms lying parallel to the direction of introduction. In doing so, however, it must be ensured that the securing function of the clip, i.e. the holding-together of the wound margins, is guaranteed and that the skin clip cannot automatically come loose from the skin. An angle of the side arms in a range of about 30° affords a sufficient holding function while at the same time substantially avoiding crushing of the skin. In addition, this setting ensures that the deeper parts of the dermis lie firmly against one another, which favors effective wound-healing.

Removal of the skin clip is also easier the less the side arms have been deflected from their direction of introduction. At said setting of 30° relative to the direction of introduction, the skin clip can be withdrawn using standard instruments, for example forceps, without requiring deformation of the skin clip. The skin lying between the two side arms constitutes an elastic tissue and can perform the compensatory deformations necessary for removal of the skin clip.

The cost involved in removing the skin clips is further reduced by the fact that the release of the skin clip is so easy that it does not necessarily have to be done by a surgeon or trained personnel. If appropriate, the skin clips can even be removed by the patients themselves or by other nonmedical staff. This is additionally made possible by the fact that it can be done without the need for any special instrument, which would again require the involvement of suitable personnel, for example a physician.

As a result of the reduced crushing of the skin, scar formation is markedly reduced. This type of wound treatment is therefore made available to a wider range of applications. Particularly in cases where visible wounds have to be treated, for example in the field of accident surgery or cosmetic surgery, it is now possible to treat wounds by clipping in this way, whereas clipping has in the past been avoided, for cosmetic reasons, and much more expensive treatment with suitable sutures has been performed.

A device according to the invention is advantageously designed such that it has a simple and straightforward construction and is easy to clean. In addition, the device according to the invention should be able to be sterilized and thus re-used. Furthermore, a device according to the invention should be easy to handle, i.e. without applying force, so that the operator does not have to focus his attention on actuating the device.

These advantages are achieved in a device which has a press element for pressing-in the shaping stamp, so that the force for applying the skin clip is exerted not by the operator, but by the press element, at least at the moment of fixing of the skin clip.

In a particularly simple embodiment, such a press element can be designed as a compression spring. In principle, however, other press elements can also be provided, for example electric motors, pneumatic cylinders or the like.

Furthermore, in a preferred embodiment, a restoring element is provided which restores the shaping stamp and, in the case of a press element as indicated above, acts counter to the press element. Such a restoring element can be designed in the form of a lifting spring. This embodiment is suitable in particular in combination with a compression spring as the press element.

In order not to weaken the force of the compression spring for pressing-in of the shaping stamp, the lifting spring is advantageously designed such that it can be uncoupled from the shaping stamp. In such an embodiment, the uncoupling can be done with the aid of an easily actuated release element, by which means application of the skin clip is triggered via the compression spring without the operator applying any force.

To restore the shaping stamp, a locking element is advantageously provided for locking the lifting spring on the shaping stamp, so that the restoring of the shaping stamp, and thus as it were the loading of the clip apparatus, can be carried out by the operator tensioning the lifting spring, with the locking element automatically locking on the shaping stamp. It should be noted here that this tensioning of the lifting spring is carried out when the device is not applied on the patient's skin and at a time when the attention of the operator is not diverted.

In a particularly advantageous embodiment, the release element is at the same time designed as a locking element, by which means the construction of the device is simplified.

It is also advantageous for the compression spring and/or lifting spring to be designed as leaf springs, with which an easy-to-grip handle can be obtained. The lifting spring in particular is in this case easily accessible for manual actuation without further operating elements.

In a further development of the invention, an adjusting element is also provided for adjusting the pretensioning of the compression spring and/or lifting spring. In a simple embodiment, such an adjusting element can be provided in the form of a tightening screw. Adjustment of the pressing force is of particular advantage if different clip sizes, and thus different designs of shaping stamp, can be actuated with one and the same clip apparatus. For larger skin clips and correspondingly more robust shaping stamps, a greater pretensioning is needed than for light clips with correspondingly lighter shaping stamp.

In a particular embodiment of the invention, a protective tape is also provided which is placed between skin and skin clip and is secured on the wound with the application of the skin clip. In this way, the wound margins are covered, so that the wound is protected from soiling, infections, mechanical influences, etc.

In a development of this embodiment, a magazine, for example a stack, of these protective tapes or a storage roll is provided from which the protective tape can be removed when required. A device according to the invention can remove such a protective tape automatically from the storage magazine. In the case of a storage roll, it suffices, for example, if the end of the protective tape is fixed with the first skin clip, so that the protective tape can be drawn off from the roll, as the whole clip apparatus advances, and is cut after the clipping procedure has finished.

An illustrative embodiment of the invention is depicted in the drawing and is explained in more detail below with reference to the figures, where:

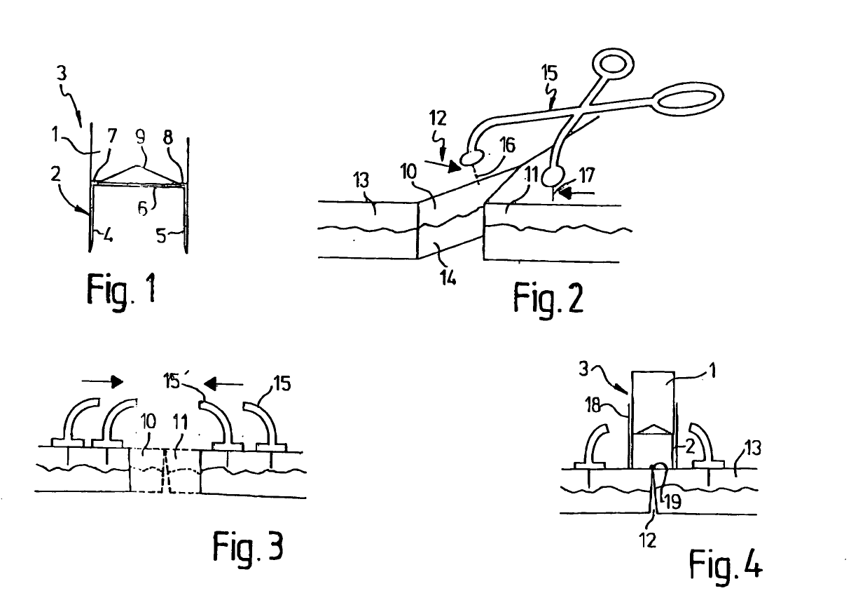

FIG. 1 shows a diagrammatic representation of a device according to the invention,

FIGS. 2-8 show individual steps in the treatment of a wound using a device according to the invention,

FIG. 9 shows a diagrammatic cross section through a device according to the invention,

FIG. 10 shows a diagrammatic representation of a front view of a device according to the invention,

FIG. 11 shows a diagrammatic outline of a clip device according to the invention before introduction of a skin clip,

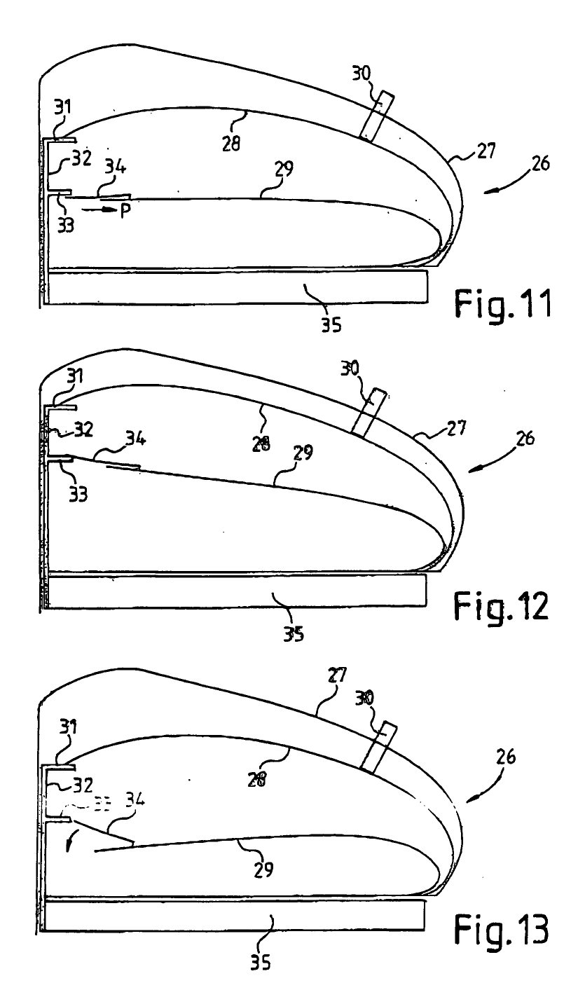

FIG. 12 shows an outline according to FIG. 11, after a clipping procedure has been triggered, and

FIG. 13 shows an outline according to FIGS. 11 and 12, with the skin clip introduced and before restoring of the shaping stamp,

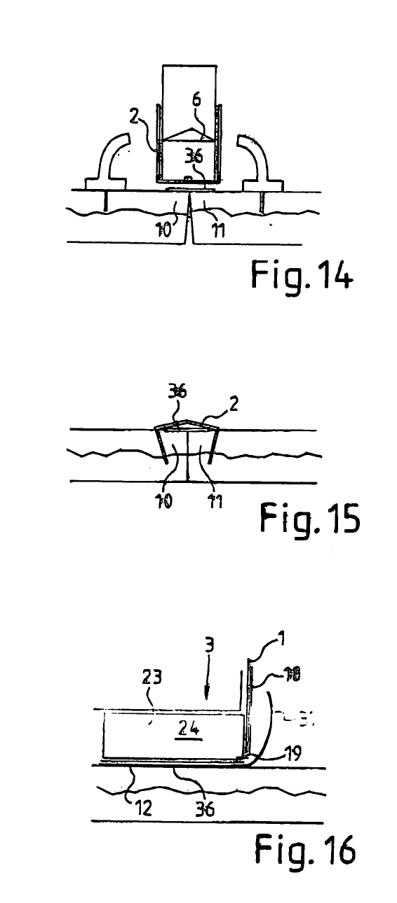

FIG. 14 shows a diagrammatic representation of a device according to the invention with a protective tape, in a front view,

FIG. 15 shows a representation of a treated wound with applied protective tape, and

FIG. 16 shows a diagrammatic side view of the device according to the invention with protective tape.

FIG. 1 shows a shaping stamp 1 and a skin clip 2 arranged below the latter, which belong to a device 3 according to the invention. The skin clip 2 comprises two side arms 4, 5 which have pointed ends and are connected to one another via an intermediate piece 6. The shaping stamp 1 corresponds in terms of its width approximately to the width of the skin clip 2 and intermediate piece 6.

In the two outer areas, the shaping stamp 1 comprises in each case a pressure-exerting shaping zone 7, 8. Between these zones, the shaping stamp 1 is provided with a V shaped contour 9. The function of the V-shaped contour 9 will be explained in detail below.

FIG. 2 shows the wound margins 10, 11 of a wound 12. The dermis 13 is shown diagrammatically here, with the fatty tissue 14 lying below it, said dermis 13 and said fatty tissue 14 gaping apart in the area of the wound.

An adaptation forceps 15 whose two tips 16, 17 penetrate into the skin serves to appose the wound margins. This state is depicted in FIG. 3. The opened position of the adaptation forceps 15 is shown here by solid black lines, while the closed position of the adaptation forceps 15′, with apposed wound margins 10, 11, is shown by broken lines.

After the wound margins have been brought together in this way, a device 3 according to the invention is used, as shown in FIG. 4. In addition to the skin clip 2 and the shaping stamp 1, the device 3 comprises a guide housing 18 for the shaping stamp 1, and a mandrel 19 which serves as a counterstay for the shaping stamp 1. In the method step according to FIG. 4, the device 3 is placed straight on the dermis 13 so that it covers the wound 12.

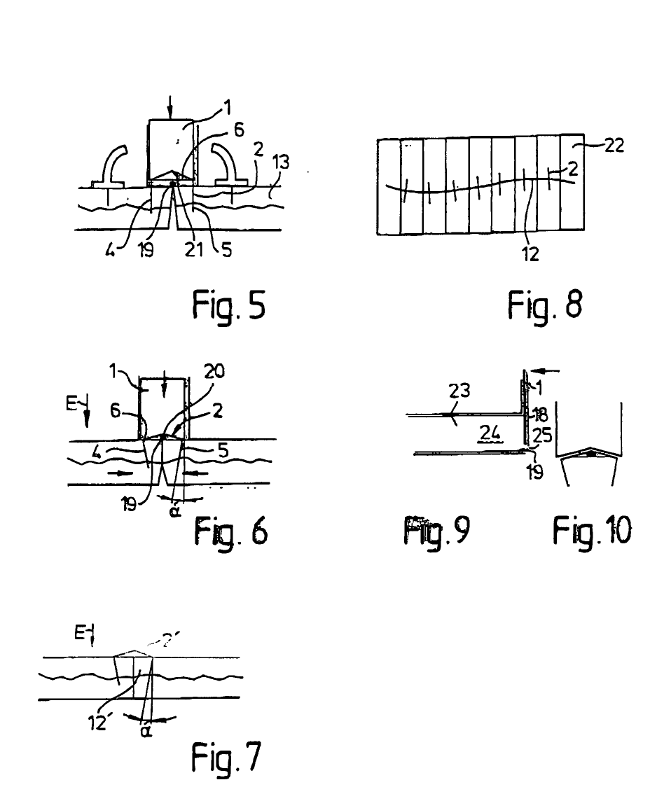

In the next method step (cf. FIG. 5), the shaping stamp 1 is depressed. This depression movement can, in case A, be effected with an impulse via a spring, as occurs in conventional “stapler” instruments, in order to achieve penetration of the vertical clip arms 4, 5 into the dermis 13 and fatty tissue 14, without this being able to able to deflect until the intermediate piece 6 of the skin clip 2 strikes against the mandrel 19 and, in the same process, the shaping stamp 1 bends the intermediate piece 6 of the skin clip 2 in such a way that a kink 20 forms above the mandrel 19.

It is also conceivable, in case B, for the vertical side arms 4, 5 to be introduced into the dermis 13 as far as the fatty tissue 14 by first pressing the clip out of the magazine until it strikes the mandrel, but without its deforming, and then for the side arms 4, 5 to be abruptly depressed by the user until the device lies on the skin. If appropriate, however, in case C with pointed clip ends, it is also possible for the clip to be introduced without impulse.

In cases B and C described above, in the next method step the shaping stamp 1 is pressed in further, as is illustrated in FIG. 6. Here, the intermediate piece 6 of the skin clip 2 is bent in such a way that a kink 20 forms above the mandrel 19. By means of this kink, the side arms 4, 5 are deflected toward one another from the direction of introduction E. The V-shaped contour 9 of the shaping stamp 1 limits the deformation of the intermediate piece 6 and thus also the angle ∝ at which the side arms 4, 5 of the skin clip 2 are deflected from the direction of introduction E.

In addition, by means of the V-shaped contour 9, in combination with the mandrel 19 which acts as counterstay, the actuation of the shaping stamp 1 is blocked as soon as the mandrel 19 and the intermediate piece 6 lying above it strike against the apex 21 of the V-shaped contour 9 of the shaping stamp 1.

In the impulse method (case A), the driving-in of the side arms 4, 5 and the deformation 20 are effected in a single operation. In this state, the skin clip 2 is already in its final state and final position, so that the device 3 can be withdrawn.

In FIG. 7, the closed wound 12′ is accordingly shown with the skin clip 2′ in its final shape. By means of the deviation ∝ from the direction of introduction E, the skin clip 2 cannot automatically come loose.

To secure against automatic loosening of this kind, it is additionally possible for adhesive strips 22 to be arranged over the skin clips 2 in the area of the wound 12, as can be seen in FIG. 8.

FIG. 9 illustrates the delivery of skin clips from a clip magazine 23. A multiplicity of skin clips are in this case supplied via a magazine guide 24 and, for example under spring pressure (not shown in more detail) are pushed in the direction of the shaping stamp 1. In the view according to FIG. 10, the shaping stamp 1 is pressed in so that it lies just above the mandrel 19. Between the guide housing 18, for the shaping stamp 1, and the mandrel 19 there is an outlet slit 25 through which the skin clips 2 (not shown in detail in this view) can leave the device 3.

By virtue of the fact that the side arms 4, 5 of the skin clip 2 deviate from the direction of introduction by a lesser deviation ∝ than in conventional clip apparatuses, a device 3 according to the invention ensures that crushing of the skin is reduced, if not completely avoided, and the skin clips 2 can be withdrawn without a special instrument.

In addition to this, the main deformation of the skin clip 2 takes place above and outside the skin, so that in this way too the risk of crushing is reduced. Moreover, the movement of the shaping stamp 1 onto the mandrel 19 ensures that the pressure needed to deform the clip 2 is not transmitted to the skin. In this way too, the risk of crushing is avoided during deformation.

The pressure for deforming the skin clip is completely independent of the pressure with which the operator presses the device onto the skin. The contact pressure on the skin must ultimately only be so great that the skin clip 2 can penetrate into the skin. As has been described, this penetration could also be achieved by means of a spring impulse.

The outline according to FIG. 11 shows a device 26 with an outer frame 27 in which a compression spring 28 and a lifting spring 29 are mounted. An adjusting screw 30 in the frame 27 is used to adjust the pretensioning of the compression spring 28.

The compression spring 28 is fitted on an upper crosspiece 31 of the shaping stamp 32. A locking projection 33 is provided for locking the release element 34. The release element 34 is secured movably on the lifting spring 29.

A clip magazine 35 is also situated on the underside of the device 26.

The device 26 outlined functions as follows. In the position shown in FIG. 11, the spring force of the compression spring 28 is overcompensated by the spring force of the lifting spring 29, so that the shaping stamp 32 is situated in its upper position above the clip magazine 35. By releasing the release element 34, for example by a displacement in the direction of the arrow P, the lifting spring 29 is uncoupled from the shaping stamp 32 so that the compression spring 28 is able to press the shaping stamp 32 downward via the crosspiece 31. In doing so, a clip is ejected from the clip magazine 35 and introduced into the skin (not shown in more detail) lying below this.

After the skin clip has been introduced into the skin, the clip device 26 is in the state according to FIG. 13. By means of the pretensioning of the lifting spring 29, the release element 34 is now brought back down to the locked position. The release element 34 is in this case mounted resiliently on the lifting spring 29. However, this function could also be realized, for example, by a flexible release element 34 or locking projection 33. Once the release element 34 is again situated in the locked position, it is able, via the lifting spring 29 and locking projection 33, to lift the shaping stamp 32 counter to the force of the compression spring 28, so that the position according to FIG. 11 is once more obtained.

This design using leaf springs permits a lightweight and straightforward construction which is easy to clean. In addition, this design affords a clip apparatus which can be sterilized and thus re-used.

FIG. 14 shows a view corresponding to FIG. 5, with a protective tape 36 now placed underneath the intermediate piece 6 of the skin clip 2 over the wound margins 10, 11. After introduction of the skin clip 2 (cf. FIG. 15), the protective tape 36 is fixed together with the wound margins 10, 11.

FIG. 16 shows a view corresponding to FIG. 9, with the protective tape 36 now fitted on the wound between the device 3 according to the invention and the wound 12. The free end 37 of the protective tape 36 is bent upward and can, for example, end in a storage roll (not shown in more detail) lying above.

By virtue of the very gentle clipping with a device according to the invention, this device can be used in applications where, in the past, clipping has been avoided, for example in cosmetic surgery or in the treatment of wounds in visible areas and in other cosmetically sensitive areas, for example the face.

In addition, the device according to the invention is of a comparatively simple construction, so that clip apparatuses and skin clips of different sizes can easily be made available.

### List of Reference Numbers

1 shaping stamp

2 skin clip

3 device

4 side arm

5 side arm

6 intermediate piece

7 shaping zone

8 shaping zone

9 v-shaped contour

10 wound margin

11 wound margin

12 wound

13 dermis

14 fatty tissue

15 adaptation forceps

16 tip

17 tip

18 guide housing

19 mandrel

20 kink

21 apex

22 adhesive strip

23 clip magazine

24 magazine guide

25 outlet slit

26 device

27 frame

28 compression spring

29 lifting spring

30 adjusting screw

31 crosspiece

32 shaping stamp

33 locking projection

34 release element

35 clip magazine

36 protective tape

37 end

1. A device for the introduction and shaping of skin clips with a clip apparatus which has a shaping stamp, and with at least one skin clip which, in the initial state, has two side arms which are parallel to each other and to the direction of introduction, and an intermediate piece connecting the side arms, characterized in that, in the shaped final state of the skin clip (2), the deviation ∝ of the side arms (4, 5) from the direction of introduction E is less than 90°.

2. The device as claimed in claim 1, characterized in that a deformation of the intermediate piece (6) is provided.

3. The device as claimed in one of the preceding claims, characterized in that a counterstay (19) is provided.

4. The device as claimed in one of the preceding claims, characterized in that two shaping zones (7, 8) are provided on the shaping stamp (1).

5. The device as claimed in one of the preceding claims, characterized in that the counterstay (19) is designed as a mandrel.

6. The device as claimed in one of the preceding claims, characterized in that the shaping stamp (1) has a V-shaped contour (9).

7. The device as claimed in one of the preceding claims, characterized in that the skin clip (2) in the initial state has a straight intermediate piece (6) and in the shaped state has a kinked intermediate piece (6).

8. The device as claimed in one of the preceding claims, characterized in that a magazine (23) for a plurality of skin clips (2) is provided.

9. The device as claimed in one of the preceding claims, characterized in that the deviation ∝ of the two side arms (4, 5) from the direction of introduction E is less than 45°.

10. The device as claimed in one of the preceding claims, characterized in that the deviation ∝ of the side arms (4, 5) from the direction of introduction E corresponds approximately to an angle of 30°.

11. The device as claimed in one of the preceding claims, characterized in that a press element (28) is provided for pressing-in the shaping stamp (32).

12. The device as claimed in one of the preceding claims, characterized in that a restoring element (29) is provided for restoring the shaping stamp (32).

13. The device as claimed in one of the preceding claims, characterized in that the press element is a compression spring (28) and/or the restoring element is a lifting spring (29).

14. The device as claimed in one of the preceding claims, characterized in that the lifting spring (29) can be uncoupled from the shaping stamp (32).

15. The device as claimed in one of the preceding claims, characterized in that a release element (34) is provided for uncoupling the lifting spring (29) from the shaping stamp (32).

16. The device as claimed in one of the preceding claims, characterized in that a locking element (33, 34) is provided for locking the lifting spring (29) on the shaping stamp (32).

17. The device as claimed in one of the preceding claims, characterized in that the release element (34) is at the same time a locking element.

18. The device as claimed in one of the preceding claims, characterized in that the compression spring and/or lifting spring (28, 29) is/are designed as a leaf spring.

19. The device as claimed in one of the preceding claims, characterized in that an adjusting element (30) is provided for adjusting the pretensioning of the compression spring and/or lifting spring (28, 29).

20. The device as claimed in one of the preceding claims, characterized in that a protective tape (36) is provided which is arranged between skin and skin clip.

21. The device as claimed in one of the preceding claims, characterized in that a storage magazine for the protective tape (36) is provided.

22. A skin clip device for fixing wound margins by introduction and shaping of skin clips, with a clip apparatus
which has a shaping stamp and a magazine for a plurality of skin clips which, in the initial state, have two straight side arms which are parallel to each other and parallel to a straight direction of introduction into the tissue to be clipped, and an intermediate piece connecting the side arms,
means being provided for introduction of the perpendicular side arms without deformation of the skin clip until it strikes against a counterstay, and for subsequent deformation, the deviation ∝ of the side arms from the direction of introduction E in the shaped final state being less than 45°, and
a compression spring being provided for pressing-in the shaping stamp, and a lifting spring as restoring element for the shaping stamp,
characterized in that
a release element for uncoupling the lifting spring from the shaping stamp and a locking element for locking the lifting spring on the shaping stamp after introduction of the skin clip are provided for the restoring operation.

23. The device as claimed in claim 22, characterized in that a deformation of the intermediate piece (6) is provided.

24. The device as claimed in one of the preceding claims, characterized in that two shaping zones (7, 8) are provided on the shaping stamp (1).

25. The device as claimed in one of the preceding claims, characterized in that the counterstay (19) is designed as a mandrel.

26. The device as claimed in one of the preceding claims, characterized in that the shaping stamp (1) has a V-shaped contour (9).

27. The device as claimed in one of the preceding claims, characterized in that the skin clip (2) in the initial state has a straight intermediate piece (6) and in the shaped state has a kinked intermediate piece (6).

28. The device as claimed in one of the preceding claims, characterized in that a magazine (23) for a plurality of skin clips (2) is provided.

29. The device as claimed in one of the preceding claims, characterized in that the deviation ∝ of the side arms (4, 5) from the direction of introduction E corresponds approximately to an angle of 30°.

30. The device as claimed in one of the preceding claims, characterized in that the release element (34) is at the same time a locking element.

31. The device as claimed in one of the preceding claims, characterized in that the compression spring and/or lifting spring (28, 29) is/are designed as a leaf spring.

32. The device as claimed in one of the preceding claims, characterized in that an adjusting element (30) is provided for adjusting the pretensioning of the compression spring and/or lifting spring (28, 29).

33. The device as claimed in one of the preceding claims, characterized in that a protective tape (36) is provided which is arranged between skin and skin clip.

34. The device as claimed in one of the preceding claims, characterized in that a storage magazine for the protective tape (36) is provided.
